(19) United States
(12) Patent Application Publication
(10) Pub. No.: US 2024/0366333 A1
(43) Pub. Date: Nov. 7, 2024

(54) VIRTUAL REALITY SYSTEM FOR ANTICIPATED VISUAL OUTCOMES OF EYE TREATMENTS

(71) Applicants: Brandon Baartman, Omaha, NE (US); Michael Greenwood, Fargo, ND (US)

(72) Inventors: Brandon Baartman, Omaha, NE (US); Michael Greenwood, Fargo, ND (US)

(21) Appl. No.: 18/653,660

(22) Filed: May 2, 2024

Related U.S. Application Data
(60) Provisional application No. 63/463,803, filed on May 3, 2023.

(51) Int. Cl. A61B 90/00

(52) U.S. Cl. CPC A61B 90/36; A61B 2090/365

(57) ABSTRACT

A vision simulation system may generate a virtual environment. The system may receive a vision profile, where the vision profile includes one or more models providing vision characteristics controlling a rendering of the virtual environment. The system may generate one or more virtual reality (VR) images representative of the virtual environment based on the vision profile. The system may provide the one or more VR images to the headset for display to a user.

### CROSS-REFERENCE TO RELATED APPLICATION

The present application claims the benefit under 35 U.S.C. § 119(e) of U.S. Provisional Application Ser. No. 63/463,803, filed May 3, 2023, entitled VIRTUAL REALITY SYSTEM FOR ANTICIPATED VISUAL OUTCOMES OF EYE TREATMENTS, naming Brandon Baartman and Michael Greenwood as inventors, which is incorporated herein by reference in the entirety.

### TECHNICAL FIELD

The present disclosure relates generally to assessing potential visual outcomes or options prior to eye surgery and, more particularly, to a virtual reality system providing simulated potential outcomes or options prior to eye surgery.

### BACKGROUND

Eye surgeries such as cataract surgery or laser vision corrective surgery are some of the most common elective surgeries performed in the United States with further anticipated growth over the next few decades. In many cases, a user is provided with multiple surgical options with potentially different visual corrections. However, users must typically make permanent vision-altering decisions based solely on conversations with a care professional, pamphlets, models, or the like. There is therefore a need to develop systems and methods for curing the above deficiencies.

### SUMMARY

In embodiments, the techniques described herein relate to a system including a controller configured to be communicatively coupled to a headset, the controller including one or more processors configured to execute program instructions causing the one or more processors to generate a virtual environment; receive a vision profile, where the vision profile includes one or more models providing vision characteristics controlling a rendering of the virtual environment; generate one or more virtual reality (VR) images representative of the virtual environment based on the vision profile; and provide the one or more VR images to the headset for display to a user.

In embodiments, the techniques described herein relate to a system, where the vision characteristics include at least one of corneal aberrations, ocular biometry measurements, scotoma measurements, optical field measurements, contrast sensitivity measurements, or accommodation range.

In embodiments, the techniques described herein relate to a system, where the vision characteristics include lens power required to produce at least one of selected emmetropic or myopic outcomes.

In embodiments, the techniques described herein relate to a system, where the vision characteristics correspond to pre-operative vision characteristics of the user.

In embodiments, the techniques described herein relate to a system, where the vision characteristics correspond to pre-operative characteristics of the user as corrected by a non-surgical device.

In embodiments, the techniques described herein relate to a system, where the non-surgical device includes at least one of eyeglasses or contact lenses.

In embodiments, the techniques described herein relate to a system, where the vision characteristics correspond to post-operative vision characteristics of the user based on a selected surgical treatment.

In embodiments, the techniques described herein relate to a system, where the vision profile is selected from a set of two or more vision profiles.

In embodiments, the techniques described herein relate to a system, where the vision characteristics are selectable by at least one of the user or a clinician.

In embodiments, the techniques described herein relate to a system, where the set of two or more vision profiles include one or more vision profiles associated with pre-operative vision characteristics of one or more eyes of the user; and one or more vision profiles associated with post-operative vision characteristics based on one or more surgical treatments.

In embodiments, the techniques described herein relate to a system, where the one or more surgical treatments include one or more intraocular lenses with different properties.

In embodiments, the techniques described herein relate to a system, where the virtual environment is selectable by at least one of the user or a clinician.

In embodiments, the techniques described herein relate to a system, where the virtual environment includes a plurality of objects at different distances from a location of the user in the virtual environment, where the vision characteristics provide distance-dependent visual properties, where representations of the plurality of objects in the one or more VR images are controlled based on associated distances from the location of the user in the virtual environment and the distance-dependent visual properties.

In embodiments, the techniques described herein relate to a system including a headset configured to display one or more VR images to a user; and a controller configured to be communicatively coupled to the headset, the controller including one or more processors configured to execute program instructions causing the one or more processors to generate a virtual environment; receive a vision profile, where the vision profile includes one or more models providing vision characteristics controlling a rendering of the virtual environment; generate one or more virtual reality (VR) images representative of the virtual environment based on the vision profile; and provide the one or more VR images to the headset for display to the user.

In embodiments, the techniques described herein relate to a system, where the vision characteristics include at least one of corneal aberrations, ocular biometry measurements, scotoma measurements, optical field measurements, contrast sensitivity measurements, or accommodation range.

In embodiments, the techniques described herein relate to a system, where the vision characteristics include lens power required to produce at least one of selected emmetropic or myopic outcomes.

In embodiments, the techniques described herein relate to a system, where the vision characteristics correspond to pre-operative vision characteristics of the user.

In embodiments, the techniques described herein relate to a system, where the vision characteristics correspond to pre-operative characteristics of the user as corrected by a non-surgical device.

In embodiments, the techniques described herein relate to a system, where the non-surgical device includes at least one of eyeglasses or contact lenses.

In embodiments, the techniques described herein relate to a system, where the vision characteristics correspond to post-operative vision characteristics of the user based on a selected surgical treatment.

In embodiments, the techniques described herein relate to a system, where the vision profile is selected from a set of two or more vision profiles.

In embodiments, the techniques described herein relate to a system, where the vision characteristics are selectable by at least one of the user or a clinician.

In embodiments, the techniques described herein relate to a system, where the set of two or more vision profiles include one or more vision profiles associated with pre-operative vision characteristics of one or more eyes of the user; and one or more vision profiles associated with post-operative vision characteristics based on one or more surgical treatments.

In embodiments, the techniques described herein relate to a system, where the one or more surgical treatments include one or more intraocular lenses with different properties.

In embodiments, the techniques described herein relate to a system, where the virtual environment is selectable by at least one of the user or a clinician.

In embodiments, the techniques described herein relate to a system, where the virtual environment includes a plurality of objects at different distances from a location of the user in the virtual environment, where the vision characteristics provide distance-dependent visual properties, where representations of the plurality of objects in the one or more VR images are controlled based on associated distances from the location of the user in the virtual environment and the distance-dependent visual properties.

In embodiments, the techniques described herein relate to a method including generating a virtual environment; generating a vision profile, where the vision profile includes one or more models providing vision characteristics controlling a rendering of the virtual environment; generate one or more virtual reality (VR) images representative of the virtual environment based on the vision profile; and provide the one or more VR images to a headset for display to a user.

It is to be understood that both the foregoing general description and the following detailed description are exemplary and explanatory only and are not necessarily restrictive of the invention as claimed. The accompanying drawings, which are incorporated in and constitute a part of the specification, illustrate embodiments of the invention and together with the general description, serve to explain the principles of the invention.

### BRIEF DESCRIPTION OF THE DRAWINGS

FIG. 1 illustrates a block diagram of a virtual reality (VR) system, in accordance with one or more embodiments of the present disclosure.

FIG. 2 illustrates a flow diagram illustrating steps performed in a method, in accordance with one or more embodiments of the present disclosure.

FIG. 3 illustrates a block diagram of various models that may be incorporated into a vision profile, in accordance with one or more embodiments of the present disclosure.

FIG. 4 illustrates a simplified schematic depicting an eye with a crystalline lens focusing on an object at a distance, in accordance with one or more embodiments of the present disclosure.

FIG. 5 illustrates a simplified schematic depicting the eye with the crystalline lens focusing on a nearby object, in accordance with one or more embodiments of the present disclosure.

FIG. 6 illustrates a plot depicting an IOL defocus curve for a particular IOL type, in accordance with one or more embodiments of the present disclosure.

FIG. 7A illustrates a simplified ray diagram of a monofocal lens, in accordance with one or more embodiments of the present disclosure.

FIG. 7B illustrates a simplified ray diagram of a toric lens, in accordance with one or more embodiments of the present disclosure.

FIG. 7C illustrates a simplified ray diagram of a multi-focal lens, in accordance with one or more embodiments of the present disclosure.

FIG. 8 illustrates blended monovision in which each eye has a monofocal lens, but the powers of the lenses are different, in accordance with one or more embodiments of the present disclosure.

FIG. 9A illustrates a simplified block diagram depicting data flow and operation of the VR system, in accordance with one or more embodiments of the present disclosure.

FIG. 9B illustrates a simplified block diagram depicting selection of a visual profile, in accordance with one or more embodiments of the present disclosure.

FIG. 10A illustrates a first screenshot of a clinician interface, in accordance with one or more embodiments of the present disclosure.

FIG. 10B illustrates a second screenshot of a clinician interface, in accordance with one or more embodiments of the present disclosure.

FIG. 10C illustrates a third screenshot of a clinician interface, in accordance with one or more embodiments of the present disclosure.

FIG. 10D illustrates a fourth screenshot of a clinician interface, in accordance with one or more embodiments of the present disclosure.

FIG. 10E illustrates a fifth screenshot of a clinician interface, in accordance with one or more embodiments of the present disclosure.

FIG. 10F illustrates a sixth screenshot of a clinician interface, in accordance with one or more embodiments of the present disclosure.

### DETAILED DESCRIPTION

Reference will now be made in detail to the subject matter disclosed, which is illustrated in the accompanying drawings. The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure.

Embodiments of the present disclosure are directed to systems and methods for providing a virtual reality (VR) simulation of different vision profiles to a user.

A VR system may generate and/or display one or more images (or a stream of images) to a user representative of a field of view of a user within a virtual environment and provide these images to a VR headset for providing the images to the user. For example, a monoscopic VR system may display a single image representing the virtual environment to both eyes of a user. As another example, a stereoscopic VR system may display a pair of stereoscopic images to different eyes of the user, where the stereoscopic images are associated with slightly different viewpoints separated by an interpupillary distance to produce a three-dimensional effect. Further, a typical VR system may render all objects within a virtual environment in focus to represent ideal or perfect vision.

As used herein, the term VR image is used to describe an image to be presented to a user via a VR headset. In some embodiments, a VR system provides a single VR image to both eyes of the user via a VR headset. In some embodiments, a VR system provides different VR images to different eyes of the user, which may be referred to as left-eye VR images and right-eye VR images.

While left-eye and right-eye VR images generated by typical VR systems differ only by the perspective within the virtual environment, left-eye and right-eye images within the present disclosure may potentially be rendered with different visual conditions. It is contemplated herein that the eyes of any particular user are typically not identical, but rather may exhibit variations that impact visual acuity or the imaging process more generally. Accordingly, some embodiments of the present disclosure may separately render left-eye and right-eye VR images to represent or in some cases simulate inter-eye variations.

In embodiments, a VR system renders VR images (or a stream of VR images) representative of a user orientation within a virtual environment based on a selected vision profile. As used herein, a vision profile is a model or a series of models that control how a virtual environment is rendered into one or more VR images to be displayed to the user in a VR headset. For example, a vision profile may include a model of one or more optical aberrations such as, but not limited to, myopia, presbyopia, or astigmatism. As another example, a vision profile may include a model of one or more physiological aspects of the eye such as, but not limited to, properties of the cornea, iris, lens, musculature, retina, blood vessels, or nerves. As another example, a vision profile may include a model of additional optics placed prior to the eye such as, but not limited to, an eyeglass lens or a contact lens. As another example, a vision profile may include a model of the vision of one or more eyes of a user prior to corrective treatment (e.g., surgical or non-surgical corrective treatment). As another example, a vision profile may include a model of the vision of one or more eyes of a user after corrective treatment.

Embodiments of the present disclosure are directed to simulating one or more visual conditions by rendering a virtual environment with a selected vision profile and displaying associated VR images to a user with a VR headset. A range of vision profiles may be generated (or generatable) that represent a range of visual conditions, where a user (or another person such as a, but not limited to, a clinician) may selectively switch between visual profiles to experience the virtual environment with associated visual conditions.

Vision profiles may be generated to simulate any of a wide variety of vision conditions to a user. It is contemplated herein that systems and methods disclosed herein may be well suited for, but is not limited to, simulating vision changes associated with different ocular treatments, natural aging processes, and/or different disease states. For example, a vision profile may represent ideal vision. As another example, visual profiles may represent the effects of various disease states or age-related deteriorations. As another example, visual profiles may represent an expected visual condition for a user after undergoing an optical treatment such as, but not limited to, implanting an intraocular lens, cataract surgery, or LASIK surgery.

In some embodiments, one or more visual profiles are generated based on biometry measurements of the user's eyes. Such a visual profile may thus represent a realistic simulation of what a user may see, particularly if separate visual profiles are generated for each eye of the user. Further, biometry measurements may be incorporated into simulated conditions such as disease states, age-related deteriorations, or optical treatments. For example, a particular surgical or non-surgical treatment may correct certain low-order corneal aberrations, but not all higher-order aberrations. Accordingly, different users may experience different visual outcomes from a particular treatment or device based on user-specific uncorrected higher-order aberrations. The systems and methods disclosed herein may thus provide user-specific estimations of visual outcomes in response to various treatment options.

It is recognized that it may be difficult or impossible for a user to fully understand the impact of various potential visual outcomes based on existing technologies. However, systems and methods disclosed herein may allow a user to interact with a virtual environment with different visual profiles and thus make a more informed decision between different treatments.

It is further contemplated that the systems and methods disclosed herein may provide superior performance than existing solutions. For example, techniques for providing static images with selectively blurred objects (e.g., based on distance-dependent aberrations, or the like) are generally described in U.S. Patent Application Publication 2020/0253468 published on Aug. 13, 2020 and U.S. Patent Application Publication 2021/0134035 published on May 6, 2021; both of which are incorporated herein by reference in their entireties. However, such techniques are limited to manipulation of an existing in-focus image. In contrast, the systems and methods disclosed herein provide on-the-fly rendering of objects in a virtual environment based on a particular vision profile. In this way, images of objects in the virtual environment are directly rendered based on the profile without the need for pixel-based manipulation. As another example, techniques for using optical elements (e.g., lenses) to manipulate light entering the eye to simulate different vision conditions are generally described in International Patent Publication WO 2012/052585 published on Apr. 26, 2012; U.S. Pat. No. 5,875,017 issued on Feb. 23, 1999; U.S. Pat. No. 8,789,951 issued on Jul. 29, 2014; U.S. Pat. No. 8,042,945 issued on Oct. 25, 2011; U.S. Pat. No. 9,693,679 issued on Jul. 4, 2017; U.S. Pat. No. 8,876,289 issued on Nov. 4, 2011; Vinas, M., Benedi-Garcia, C., Aissati, S. et al. Visual simulators replicate vision with multifocal lenses. Sci Rep 9, 1539 (2019); and Kyung-Sun Na, Seong-Jae Kim, Gahee Nam, Minji Ha, Woong-Joo Whang, Eun Chul Kim, Hyun-Seung Kim, Ho Sik Hwang; A Novel Intraocular Lens Simulator that Allows Users to Experience the World Through Multifocal Intraocular Lenses Before Surgeries. Trans. Vis. Sci. Tech. 2022; 11(3):14; all of which are incorporated herein by reference in their entireties. However, such techniques are limited by the quality and design of the optical elements and may only partially simulate a visual outcome. Further, such techniques may not simulate the impact of a surgical procedure on the accommodation range of the eye.

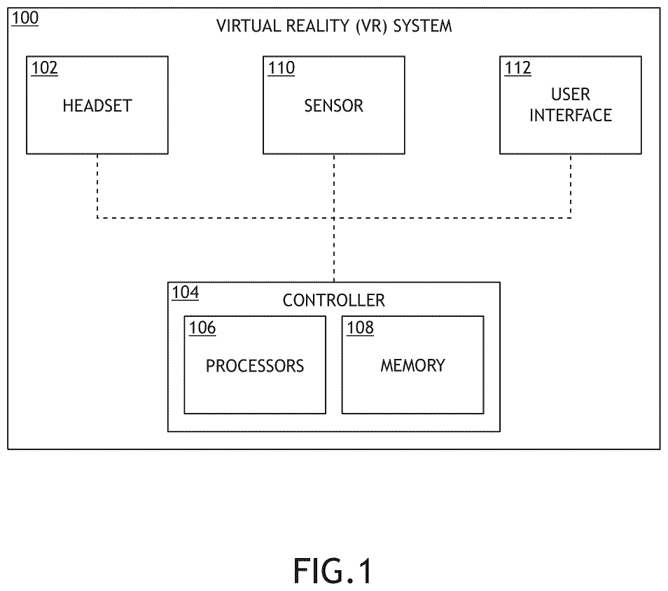

Referring now to FIGS. 1-10E, systems and methods for simulation of different visual profiles in a virtual reality environment are described in greater detail, in accordance with one or more embodiments of the present disclosure.

FIG. 1 illustrates a block diagram of a VR system 100, in accordance with one or more embodiments of the present disclosure.

In some embodiments, the VR system 100 includes a headset 102 (e.g., a VR headset) suitable for providing VR images to the eyes of a user and a controller 104 with one or more processors 106 configured to execute program instructions maintained on memory 108 (e.g., a memory medium), where the program instructions cause the processors 106 to perform various steps or tasks. For example, the processors 106 may be configured to perform steps such as, but not limited to, generating a virtual environment, generating VR images representative of a field of view of the user within the virtual environment, and providing the VR images to the headset 102 for display to the user. As another example, the processors 106 may be configured to generate one or more visual profiles representative of a user's vision under different conditions, where a visual profile may incorporate any combination of pre-operative characteristics, post-operative characteristics after a selected treatment, or using non-surgical corrective devices (e.g., eyeglasses, contact lenses, or the like). In this way, the VR images provided to the headset 102 may be generated based on a selected visual profile to simulate vision of the virtual environment with this visual profile to the user.

The headset 102 may include any components or combination of components suitable for providing VR images to a user and thus operating to immerse the user in a virtual environment based on one or more VR images. For example, the headset 102 may include any commercially available VR headset such as, but not limited to, the HP Reverb G2, the HTC Vive Pro 2, the Valve Index, the Sony PlayStation VR2, the Meta Quest 2, or the Meta Quest Pro. As another example, the headset 102 may be custom fabricated.

In some embodiments, the headset 102 is configured to be worn on the head of a user and further configured to at least partially cover the user's eyes (e.g., obstruct the user's line of vision) such that the user may only see the provided VR images. The headset 102 may further include one or more light sources and one or more projection optics configured to direct VR images to the user's eyes. It is contemplated herein that the headset 102 may utilize any display or projection technology known in the art suitable for providing VR images. For example, the headset 102 may include one or more display screens as light sources and one or more stereoscopic lenses configured to separately direct different images to the left and right eyes of the user. As another example, the headset 102 may include separate displays and separate optics for each eye of the user. As another example, the headset 102 may include a retina projection system with one or more lasers or light emitting diodes (LEDs) as light sources and projection optics to project images directly onto the user's retinas. Such a retina projection system may project full two-dimensional (2D) images onto the user's retinas or may build up an image through point-by-point or line-by-line scanning.

In some embodiments, the headset 102 includes one or more shutters to selectively block or pass light to a particular eye. In this way, the headset 102 may selectively direct light (e.g., a corresponding image of a spectroscopic image pair) to both eyes or a left or right eye individually.

In some embodiments, the VR system 100 includes one or more sensors 110 to monitor the orientation and/or position of the user, which may be provided to the controller 104. These sensors 110 may be integrated within the headset 102 or provided as stand-alone devices. In this way, the controller 104 may update the VR images (e.g., the stream of VR images) to reflect changes in the orientation and/or the position of the user. For example, the one or more sensors 110 may monitor a gaze direction (e.g., head orientation and/or eye tracking sensors) of the user such that the controller 104 may update the field of view associated with the virtual environment provided by the VR images accordingly. As another example, the one or more sensors 110 may monitor a position of the user such that the controller 104 may update the position of the user in the virtual environment provided by the VR images accordingly. As another example, the one or more sensors 110 may monitor positions of various additional body parts such as, but not limited to, hands or feet of the user such that the controller 104 may provide a visualization of the user's body or an avatar in the virtual environment that mimics real motions of the user.

The one or more sensors 110 may include any type of sensors known in the art suitable for tracking the orientation and/or position of the user's body, or portions thereof. Further, the one or more sensors 110 may be provided as dedicated components or may be integrated into other components of the VR system 100 such as, but not limited to, the headset 102. In some embodiments, at least one sensor 110 is located in the headset 102. For example, the headset 102 may include an eye-tracking sensor 110 to monitor the eye directions of the user's eyes and/or a rotational sensor 110 to monitor a user's head direction or orientation. As another example, the headset 102 may include an outward-facing camera as a sensor 110, which may be used for a variety of purposes including, but not limited to, monitoring head orientation, monitoring hand and/or foot positions, or location monitoring. In some embodiments, at least one sensor 110 is held by the user (e.g., as part of a hand-held controller, or the like). In some embodiments, at least one sensor 110 is worn by the user (e.g., attached to or part of an item of clothing). In some embodiments, at least one sensor 110 includes a camera configured to image the user or a portion thereof while wearing the headset 102, which may be used for any type of orientation or position tracking.

In some embodiments, the VR system 100 includes a user interface 112 communicatively coupled to the controller 104. For example, the user interface 112 may include, but is not limited to, one or more desktops, laptops, tablets, and the like. As another example, the user interface 112 includes a display used to display data of the VR system 100 to a user such as, but not limited to, a user or a clinician. As an illustration, such a display may be used to display a two-dimensional version of the VR images displayed by the headset 102. The display of the user interface 112 may include any display known in the art. For example, the display may include, but is not limited to, a liquid crystal display (LCD), an organic light-emitting diode (OLED) based display, or a CRT display. As another example, the user interface 112 may include one or more input devices suitable for capturing input from the user or a clinician. As an illustration, such an input device may include a mouse, a keyboard, or a touchscreen. As another illustration, such an input device may include controls coupled with the headset 102 for interacting in a virtual environment (e.g., a gesture sensor/controller, handheld controllers, voice controllers, or the like).

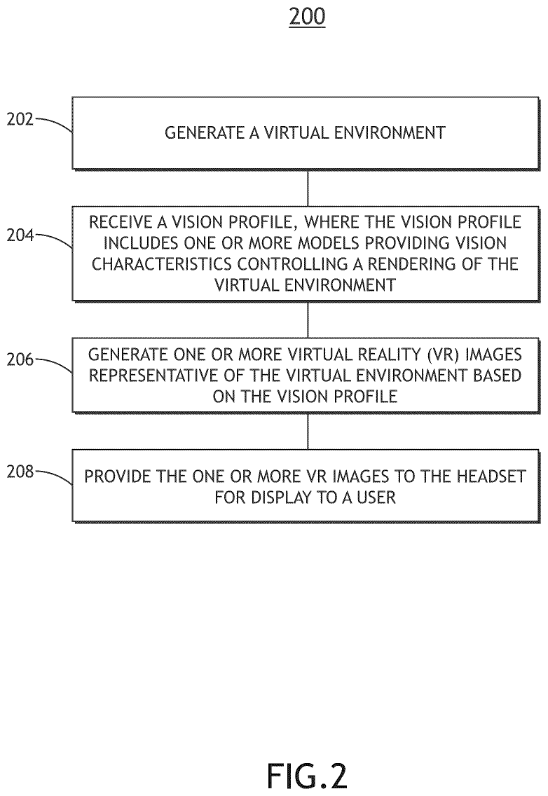

Referring now to FIGS. 2-10E, selective visualization of virtual environments with different visual profiles is described in greater detail, in accordance with one or more embodiments of the present disclosure.

FIG. 2 illustrates a flow diagram illustrating steps performed in a method 200, in accordance with one or more embodiments of the present disclosure. Applicant notes that the embodiments and enabling technologies described previously herein in the context of the VR system 100 should be interpreted to extend to the method 200. For example, the processors 106 of the controller 104 may be configured to execute program instructions causing the one or more processors 106 to implement any of the steps of the method 200. It is further noted, however, that the method 200 is not limited to the architecture of the VR system 100.

In some embodiments, the method 200 includes a step 202 of generating a virtual environment. For example, the virtual environment may correspond to a scene to be presented to the user and may include multiple objects. In some cases, at least some of the objects are manipulatable by a user. Such a scene may include, but is not limited to, a room of a house (e.g., a kitchen, a living room, or the like), an outdoor scene, or a view from an automobile. A virtual environment may generally include any number or type of objects at any locations. In some embodiments, a virtual environment includes objects at different locations such that various objects at different distances may be visible from typical locations within the virtual environment, which may be beneficial for evaluating visual outcomes associated with objects at various distances for different treatment plans.

The virtual environment may be constructed and/or stored in memory 108 using any technique known in the art. In some embodiments, the virtual environment includes information associated with various objects including, but not limited to, size, shape, orientation, weight, location in a three-dimensional space, attachment information (e.g., connections between objects), textures, colors, or graphics. In this way, images associated with a field of view from a particular location within the virtual environment may be generated.

The virtual environment may support rendering from one or more locations. Further, the virtual environment may include objects at various distances and directions from any of the locations. In this way, the user, while wearing the headset 102, may physically turn his or her head to view objects within different fields of view associated with different directions within the virtual environment.

For example, the VR system 100 may provide distance-dependent visual properties. This may mean that representations of the plurality of objects in the images are controlled based on associated distances from the location of the user in the virtual environment and the distance-dependent visual properties.

The virtual environment may support rendering in different lighting conditions such as, but not limited to, lighting intensity (e.g., outdoor daylight conditions, low-light conditions, interior lighting conditions, or the like) or lighting colors (e.g., different color temperatures and/or specific wavelengths).

The virtual environment may support user interaction with objects. For example, any objects in the virtual environment may be manipulatable by the user in the environment. As an illustration, a user may pick up a book, open a window, turn lighting on or off, or the like. Further, a user's perception of any object may change based on the rendering of the object with a particular vision profile. For example, a degree to which an object is in focus may vary as a user either manipulates the object or moves relative to the object. For instance, a user may pick up a book, observe that it is slightly blurred, and move it to a comfortable distance for reading based on a particular vision profile.

It is contemplated herein that the virtual environment may have a range of complexities or features within the spirit and scope of the present disclosure. In some embodiments, a user is limited to a single fixed location within the virtual environment. As a non-limiting illustration, the virtual environment may correspond to an interior room in which the user is seated at a table or desk. In this configuration, the user may turn his or her head to view different portions of the room from this location and/or switch between standing or seated positions. In some embodiments, a user may move within the virtual environment. For example, the user may move between a set of fixed locations within the virtual environment or may freely move to any location. In some embodiments, a user may interact with objects in the virtual environment. For example, the user may pick up a book or a menu in the virtual environment to evaluate near-distance visual outcomes.

In some embodiments, the method 200 includes a step 204 of generating (or receiving) vision profiles associated with visual characteristics of a user. In some embodiments, the method 200 includes a step 206 of generating one or more VR images representative of the virtual environment based on the vision profile. For example, the VR images may be representative of a field of view of the user in the virtual environment as rendered by the vision profile (or the models therein) based on a location of the user in the virtual environment. In some embodiments, the VR images are further based on the gaze direction of the user (e.g., as determined by sensors 110). In some embodiments, the method 200 includes a step 208 of providing the VR images to the headset for display to the user. More broadly, the step 204 may include generating multiple vision profiles associated with different visual characteristics. Further, different vision profiles may be generated and used for each eye. In this way, the method 200 may further include a step 210 of selecting a particular vision profile for each eye (or at least one) prior to generating the VR images for display by the headset.

As used herein, a vision profile is a model or a series of models that control how a virtual environment is rendered into one or more VR images to be displayed to the user in a VR headset. Further, a vision profile may include one or more models providing vision characteristics controlling the rendering of the virtual environment (e.g., into VR images for display to the user). In this way, a vision model may simulate the vision of the eye, where the models may incorporate any vision characteristics associated with how a particular eye may generate an image (e.g., aberrations, or the like).

In some embodiments, a vision profile provides distance-dependent rendering of objects such that objects within the virtual environment located at different distances from the user may be rendered differently. For example, the vision characteristics may provide distance-dependent visual properties such that objects in the virtual environment at different distances from the user in the virtual environment may be rendered differently. As an illustration, a vision profile may incorporate vision characteristics such as aberrations, myopia, presbyopia, or the like such that VR images rendered based on the vision profile may provide a realistic simulation of vision under the selected visual conditions.

In some embodiments, a vision profile includes a model that describes deviations from an ideal state.

For example, a vision profile may include a model of optical aberrations (e.g., an aberration model) such as, but not limited to, spherical aberration, astigmatism, coma, defocus, distortion, or chromatic aberration. It is contemplated herein that optical aberrations (e.g., astigmatism, defocus, and the like) are commonly used by medical practitioners to describe the biometry of a patient as well as various treatment options. For this reason, incorporating an optical aberration model into a vision profile may provide a convenient framework for tailoring the visual characteristics related to how the virtual environment is rendered and displayed to the user based on common biometry measurements.

In some embodiments, a vision profile may include a model of an optical field (e.g., a field of vision). In this way, different vision profiles may be generated with variations in this optical field to simulate various conditions such as, but not limited to, scotoma from disease states such as macular degeneration, macular hole, retinal detachment, glaucoma, or optic neuropathy.

In some embodiments, a vision profile may include a set of lens powers (e.g., optical powers). In this way, the vision profile may include various lens powers that are required to produce a selected emmetropic and/or myopic outcome for the user.

In some embodiments, a vision profile may include a model of contrast sensitivity or another metric of visual quality. In this way, different vision profiles may be generated with variations in this optical field to simulate various conditions such as, but not limited to, glaucoma, optical neuropathy, ocular surface disease, epithelial basement membrane dystrophy, keratoconus, or Fuch's corneal dystrophy.

In some embodiments, different vision profiles are generated to emphasize the impact of different lighting conditions. For example, it may be the case that certain visual artifacts (e.g., halos, starbursts, or the like) may be more prevalent or more noticeable in low-light conditions, particularly around light sources. While these effects may be generally simulated under all lighting conditions, it may be desirable in some applications to present such visual artifacts (e.g., in the form of a visual profile) only when expected to be present. Any suitable trigger may be used to switch between such different visual profiles such as, but not limited to, when light intensity is at or below a threshold value.

In some embodiments, a vision profile includes models of one or more aspects of the optical system provided by the eye. In this way, the virtual environment may be rendered in a manner that at least partially mimics the operation of the eye. Such an approach may be suitable for, but not limited to, modeling vision based on physiological considerations, which may relate to the root causes associated with various optical aberrations or deviations from ideal vision conditions more generally. Such an approach may also allow for accurate modeling of various treatment options based on their specific impact to specific portions of the eye.

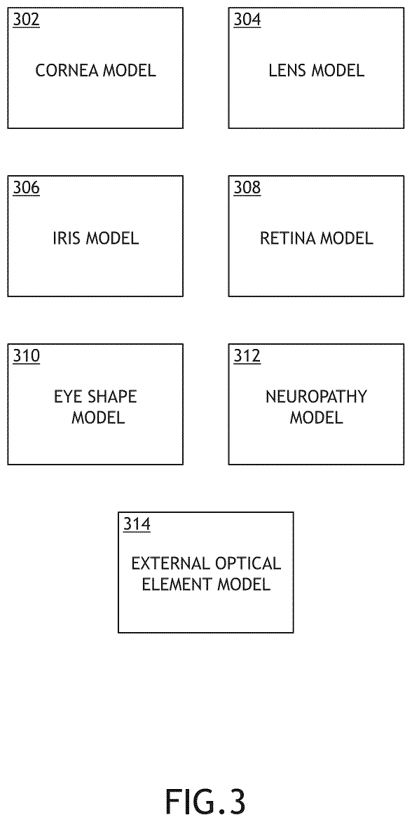

FIG. 3 illustrates a block diagram of various models that may be incorporated into a vision profile, in accordance with one or more embodiments of the present disclosure. For example, a vision profile may include, but is not limited to, any combination of the models shown in FIG. 3.

In some embodiments, a vision profile includes a cornea model 302, which may incorporate any combination of optical or physiological characteristics of the cornea. For example, the cornea model 302 may incorporate optical characteristics such as, but not limited to, an optical power, optical aberrations (e.g., astigmatism, or the like), or transmissivity characteristics. As another example, the cornea model 302 may incorporate a shape and/or thickness of the cornea.

In some embodiments, a vision profile includes a lens model 304, which may incorporate any combination of optical or physiological characteristics of the crystalline lens in the eye (e.g., the lens with elasticity providing varying optical power to enable focusing on objects at different distances). For example, the lens model 304 may incorporate a range of optical powers achievable by the crystalline lens, which may vary based on factors such as, but not limited to, age or the elasticity of the crystalline lens. As another example, the lens model 304 may incorporate the elasticity of the crystalline lens itself. As another example, the lens model 304 may incorporate optical aberrations induced by the crystalline lens. As another example, the lens model 304 may incorporate transmissivity characteristics of the crystalline lens. For instance, the condition of cataracts may result in decreased transmissivity of light through the crystalline lens.

In some embodiments, a vision profile includes an iris model 306, which may incorporate any combination of optical or physiological characteristics of the iris. For example, the iris model 306 may incorporate a pupil size, which may be impact various aspects of vision including, but not limited to, a depth of field or an impact of certain optical aberrations. As an illustration, many optical aberrations may provide that rays from different portions of a lens (e.g., the cornea or the crystalline lens) come to a focus at different locations, which results in blur. The impact of such optical aberrations may thus depend on the size of a bundle of rays propagating through the eye, which is controlled by the iris.

In some embodiments, a vision profile includes a retina model 308, which may incorporate any combination of optical or physiological characteristics of the retina. For example, the retina model 308 may describe the functionality of various aspects of the retina including the macula or the peripheral retina. As an illustration, the retina model 308 may describe the operation (or deficiencies in operation) of the various photoreceptors (e.g., rods and cones) that convert light incident on the retina to electrical signals for transmission to the brain via the optic nerve. In this way, the effects of abnormalities, disease, or damage to the retina may be incorporated into the vision profile. For instance, the retina model 308 may be used to simulate color blindness (e.g., color deficiency) related to the operation of various cone cells in the retina.

In some embodiments, a vision profile includes an eye shape model 310, which may incorporate any combination of optical or physiological characteristics of the eye as a whole. For example, the eye shape model 310 may include physiological aspects of the eye such as, but not limited to, an eye length (e.g., a distance between the crystalline lens and the retina), or a retina shape (e.g., a curvature of the retina). It is contemplated herein that the ability of an eye to bring a particular object into focus on the retina may depend on multiple factors such as, but not limited to, the eye length, the shape of the retina, and the achievable optical power associated with the combination of the cornea and the crystalline lens.

In some embodiments, a vision profile includes a neuropathy model 312, which may incorporate any combination of optical or physiological characteristics of the optic nerve or other systems related to the neurological transfer of an image on the retina to the brain. For example, the neuropathy model 312 may describe damage to the optic nerve or other neurological pathways that may induce how an image that is optically generated on the retina is received and/or perceived by the user.

In some embodiments, a vision profile includes an external optical element model 314, which may describe the presence of any additional optical elements external to the eye such as, but not limited to, eyeglasses (prescription eyeglasses, contact lenses, non-prescription optics, sunglasses, or the like). In this way, different vision profiles may be used to simulate variations in parameters of such additional optical elements. As an illustration, a user may evaluate different parameters of prescriptive lenses through different vision profiles in which parameters in this external optical element model 314 are varied.

Considering the various models in FIG. 3, it is contemplated herein that the perception of vision by a user is a complex function of various components that form the eye and/or additional external components between the eye and an object. Accordingly, a vision profile may include models to specifically describe any such optical component or combination of optical components.

As an illustration, the focusing power of the eye may be attributed to the combined action of the cornea and the crystalline lens, where the cornea has a fixed optical power and the crystalline lens may provide an adjustable optical power (e.g., may provide accommodation). As a result, a range of optical powers that an eye may achieve may be impacted by properties of the cornea as well as various physiological systems associated with accommodation such as, but not limited to, the shape of the crystalline lens, the flexibility of the crystalline lens, or the strength of musculature surrounding the crystalline lens. As depicted in FIG. 3, the use of multiple models to separately describe the operation of different components of the eye (e.g., a cornea model 302, a lens model 304, an eye shape model 310, or a combination thereof) may enable the user to generate different vision profiles to separately evaluate variations caused by each of these components.

As another illustration, the ability of an eye to bring a particular object into focus on the retina may be influenced by various physiological systems such as, but not limited to, the range of optical powers that the eye may achieve, the length of the eye (e.g., a separation distance between the lens and the retina), or the pupil size. For example, myopia may occur when the eye provides an image plane (e.g., a plane at which light from a particular object is in focus) prior to the retina due to any combination of the length of the eye or the range of optical powers achievable by the cornea and lens. As another example, presbyopia may occur when the eye provides an image plane after the retina due to any combination of the length of the eye or the range of optical powers achievable by the cornea and lens and is a common condition of aging due to hardening of the lens. As depicted in FIG. 3, the use of a cornea model 302, a lens model 304, an eye shape model 310, an iris model 306, or a combination thereof may enable the user to generate different vision profiles to separately evaluate variations caused by each of these components.

As another illustration, the use of multiple models as depicted in FIG. 3 may enable the generation of accurate models that simulate various diseases of the eye based on the physiological impact on the eye. For example, the impact of various stages of cataracts may be simulated by creating different vision profiles in which the transmissivity of the crystalline lens in the lens model 304 is varied while other aspects of the vision may remain the same. As another example, the impact of various stages of glaucoma may be simulated by manipulating a neuropathy model 312 and/or a retina model 308 while other aspects of vision may remain the same. As another example, scotoma from disease states such as macular degeneration, macular hole, or retinal detachment may be simulated using relevant models such as, but not limited to, a retina model 308 or a neuropathy model 312. As another example, the impact of visual field loss and loss of contrast sensitivity from disease states such as glaucoma or optic neuropathy may be simulated using relevant models such as, but not limited to, a retina model 308 or a neuropathy model 312. As another example, loss of contrast sensitivity and visual quality from corneal disorders such as ocular surface disease, epithelial basement membrane dystrophy, keratoconus, or Fuch's corneal dystrophy may be simulated using relevant models such as, but not limited to, a cornea model 302. More broadly, targeted simulations of various stages of any disease may be provided by generating vision profiles associated with relevant models associated with the perception of vision.

In some embodiments, a vision profile is generated that simulates an expected outcome associated with a corrective procedure. For example, and as will be described in greater detail below, different vision profiles may be created that correspond to a preliminary state of the user's eyes (e.g., prior to surgery and without corrective lenses) as well as for a variety of corrective options such as, but not limited to, different non-surgical corrective lens options (e.g., simple lens, bi-focal lens, tri-focal lens, progressive lens, or the like) and/or different surgical options (e.g., corneal surgical options, different refractive endpoint targets, different IOL options, or the like).

Such vision profiles may incorporate any type of model such as, but not limited to, models of specific optical phenomena (e.g., an aberration model, an optical field model, a contract sensitivity model, or the like), models based on physiological components of the eye (e.g., as depicted in FIG. 3), or a combination thereof. However, it is contemplated herein that the use of multiple models based on physiological components of the eye (e.g., as depicted in FIG. 3) may provide flexible and accurate simulations by modeling the impact on the appropriate physiological component. For example, in the case of corneal surgical options (e.g., LASIK), the impact of various treatment options may be simulated by changing the cornea model 302 while leaving other models untouched. As another example in the case of cataracts surgery, the impact of various IOL models may be simulated by changing the lens model 304 while leaving other models intact.

In a general sense, information associated with visual pathologies may be generic or may be personalized to the user. For example, generic pathology data may include parameters suitable for modeling known lens aberrations of a generic human eye, whereas personalized information may be generated by measurements and/or tests on the user. In some cases, biometry data (e.g., optical biometry data, biometric data, or the like) may be generated to determine various low or high order aberrations.

In some embodiments, one or more models associated with vision profiles are tailored to a particular user based on one or more biometry measurements of one or both eyes of a user. For example, a different cornea model 302 may be used for each eye that captures the associated corneal aberrations and/or corneal optical power based on biometry measurements.

Any type of biometric data associated with one or both eyes of a user may be incorporated into a vision profile (or any model therein). For example, biometric data may include, but is not limited to, keratometry (e.g., corneal power in the steep (K1) and flat (K2) axis), an amount of anterior corneal astigmatism (the delta of K1−K2), pupil size, or corneal higher order aberrations.

Table 1 below illustrates exemplary biometric data in a right eye of a user, in accordance with one or more embodiments of the present disclosure.

TABLE 1

| Patient XZY | Right eye |
|---|---|
| Astigmatism | |
| Steep K | 44.49 |
| Flat K | 43.63 |
| Anterior corneal cyl | 0.86 |
| Anterior Axis | 112 |
| Posterior corneal cyl | 0.4 |
| Posterior Axis | 90 |
| Anticipated Post Op Cylinder | 0.52 |
| Post op cylinder axis | 105 |
| Higher-order Aberrations | |
| Coma | 0.134 |
| Trefoil | 0.155 |
| Spherical Aberration | 0.12 |
| Total | 0.266 |
| Pupil size | |
| mm | 4 |

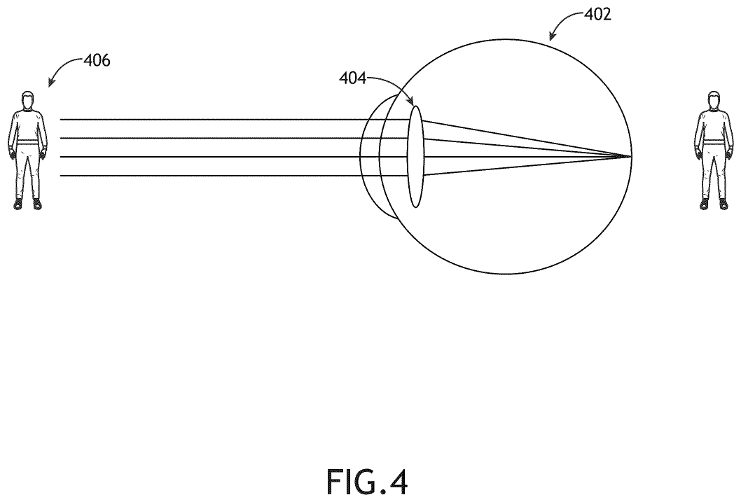

Referring now to FIGS. 4-8, simulations of expected visual outcomes associated with implantation of an IOL (e.g., as part of cataracts surgery, or the like) are described in greater detail, in accordance with one or more embodiments of the present disclosure. In particular, the impacts of different lens designs and various tradeoffs that may be incorporated into different visual profiles for simulation are described in greater detail, in accordance with one or more embodiments of the present disclosure. It is to be understood that FIGS. 4-8 are merely illustrative and should not be interpreted as limiting the scope of the present disclosure. Rather, the systems and methods disclosed herein may be used to simulate any type of visual condition.

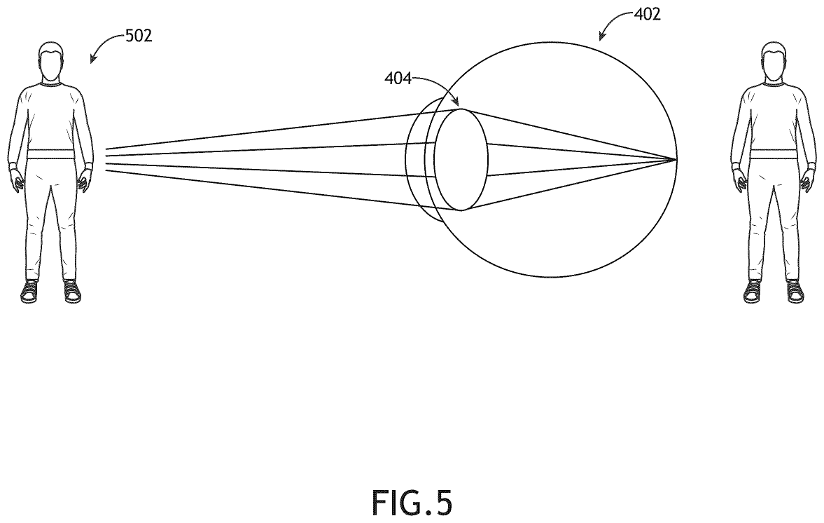

As described previously herein, certain ocular treatments such as IOLs may partially or fully reduce the accommodation range in eye and thus impact the ability of the eye to focus on objects at different distances. FIG. 4 illustrates a simplified schematic depicting an eye 402 with a crystalline lens 404 focusing on an object 406 at a distance, in accordance with one or more embodiments of the present disclosure. FIG. 5 illustrates a simplified schematic depicting the eye 402 with the crystalline lens 404 focusing on a nearby object 502, in accordance with one or more embodiments of the present disclosure. Note the change in the shape of the crystalline lens 404 that modifies the refracting power of the eye needed to properly focus objects at various distances. In cases where treatment options partially or fully remove the accommodation range, this natural ability to focus on objects at different distances is diminished. Accordingly, various lens designs or treatment options may be considered that incorporate different visual tradeoffs. It is contemplated herein different vision profiles for any of these options may be provided to a user (e.g., via the VR system 100 and/or through the method 200) to allow the user to make an informed decision.

In some embodiments, different visual profiles may be generated based on different intraocular lens (IOL) options, as well as for pre-operative conditions with or without non-surgical treatments (e.g., eyeglasses, contact lenses, or the like). Further, the ability of an eye to accommodate (e.g., change shape) to focus on objects at different distances may be reduced or eliminated. Further, certain treatment options may correct some aberrations but not others (e.g., may correct some lower-order aberrations but not some higher-order aberrations) such that the visual outcomes may reflect residual uncorrected aberrations. In such cases, it may be particularly beneficial to tailor the vision profiles to a particular user based on biometry measurements such that the vision profiles may provide a realistic simulation of the expected outcomes of the particular user.

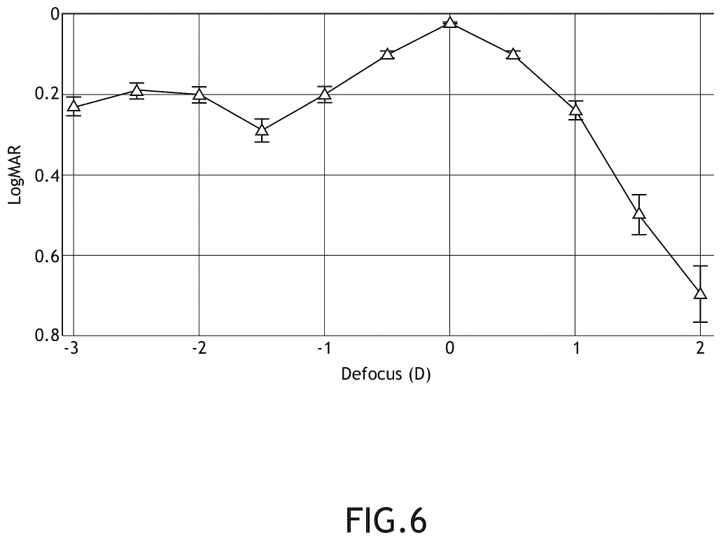

FIG. 6 illustrates a plot depicting an IOL defocus curve for a particular IOL type, in accordance with one or more embodiments of the present disclosure. In this plot, the X axis depicts an amount of defocus, with 0 being no defocus in either direction. For example, moving left (negative) simulates the performance of a lens as an object in the virtual environment is moved closer, noting a drop in LogMAR vision performance. Moving right from 0 (positive) simulates the performance of the lens as the object is moved further away from the user in the virtual environment.

It is contemplated herein that different vision profiles associated with different IOL designs may incorporate particular defocus curves (or any other simulation of visual performance) such that a user may selectively switch between the different vision profiles to experience or evaluate the expected visual outcomes for the different IOL designs.

The defocus properties depicted in FIG. 6 may be incorporated into any model of a vision profile associated with the particular IOL. For example, the defocus properties depicted in FIG. 6 may be incorporated into a lens model 304 as depicted in FIG. 3. In cases where user biometry is used to tailor the vision profile to a particular user, this configuration allows for selective modification of the lens model 304 while keeping other user-specific biometry data (e.g., corneal aberration data in a cornea model 302) untouched, which may provide a realistic simulation of an expected visual outcome for that particular user. As another example, the defocus properties depicted in FIG. 6 may be incorporated into an optical aberration model that may describe vision at a higher level.

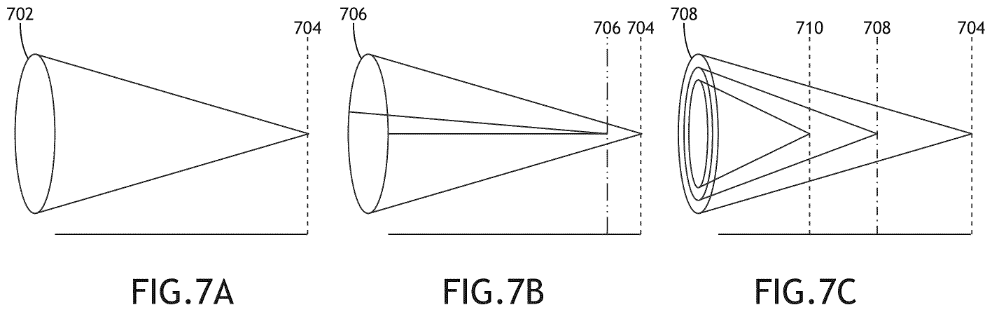

FIGS. 7A-7C depict three different lens designs. FIG. 7A illustrates a simplified ray diagram of a monofocal lens 702, in accordance with one or more embodiments of the present disclosure. Such a lens may have a single focal length 704. With this design, objects at a selected distance may be in focus on the retina, while objects away from this selected distance may be increasingly blurred. FIG. 7B illustrates a simplified ray diagram of a toric lens 706, in accordance with one or more embodiments of the present disclosure. Such a lens may have different focal lengths 704, 706 for light rays along different directions and may thus be used to correct for astigmatism. FIG. 7C illustrates a simplified ray diagram of a multi-focal lens 708, in accordance with one or more embodiments of the present disclosure. This design may provide multiple focal points 704, 708, 710 such that objects at multiple focal distances may be simultaneously in focus.

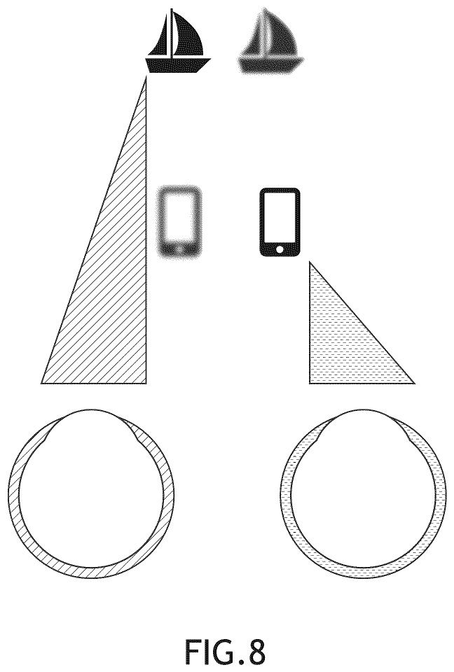

As described previously herein, different vision profiles may be generated for each eye of a user. In this way, different VR images may be separately generated for each eye based on different renderings of the virtual environment using the different visual profiles. Such a configuration may be suitable for, but not limited to, simulating blended monovision. FIG. 8 illustrates blended monovision in which each eye has a monofocal lens, but the powers of the lenses are different, in accordance with one or more embodiments of the present disclosure. For example, FIG. 8 depicts a configuration in which a left eye has a monofocal lens configured to provide in-focus images of distant objects (here represented by the sailboat), while the right eye has a monofocal lens configured to provide in-focus images of near objects (here represented by a phone). Again, a vision profile may be generated to simulate such a treatment plan in the virtual environment for objects at various distances.

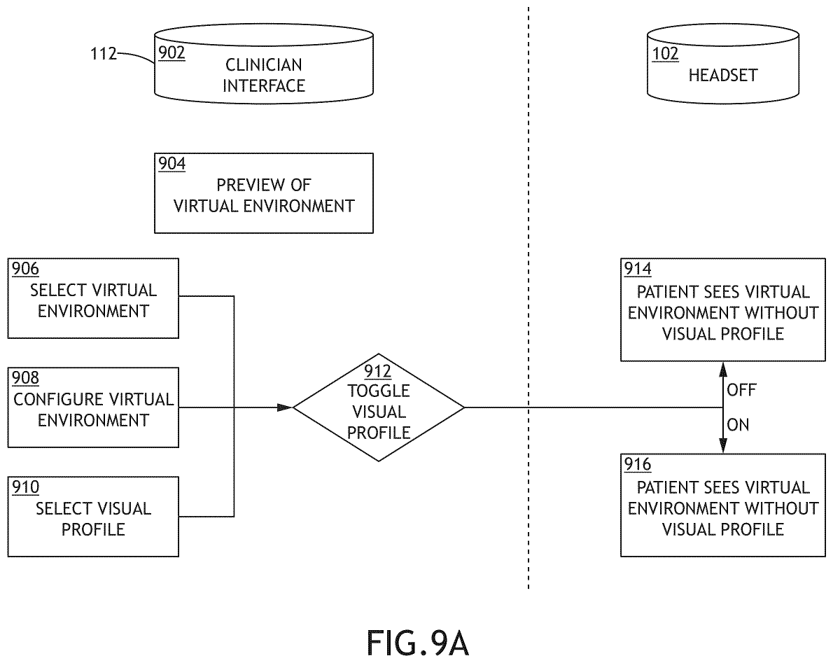

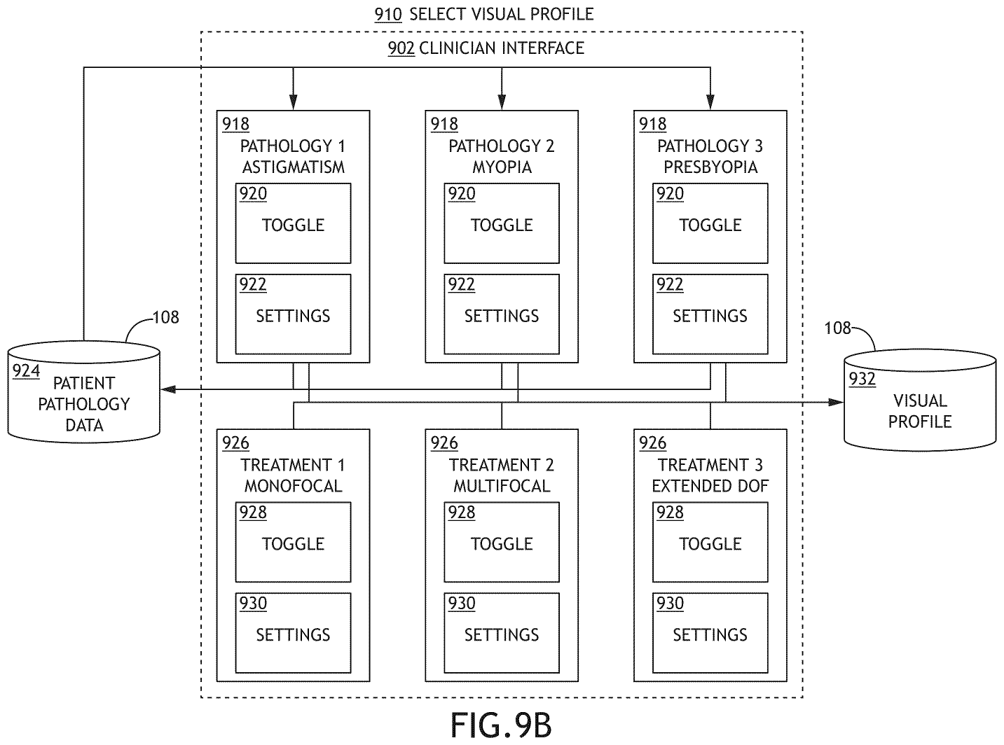

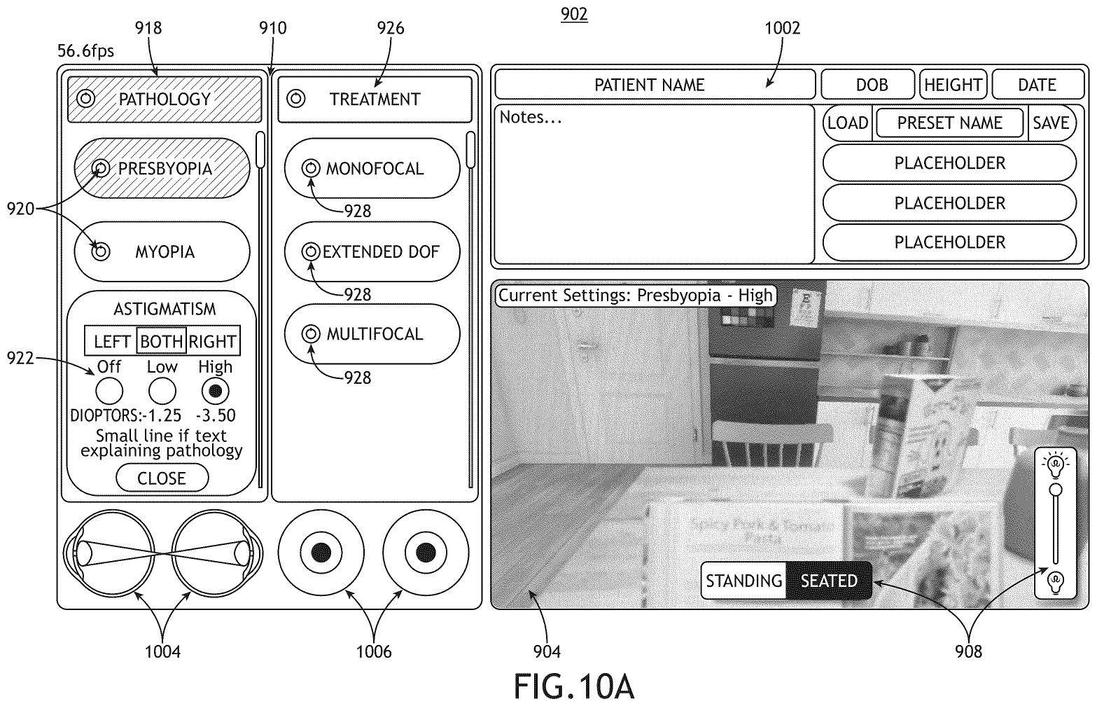

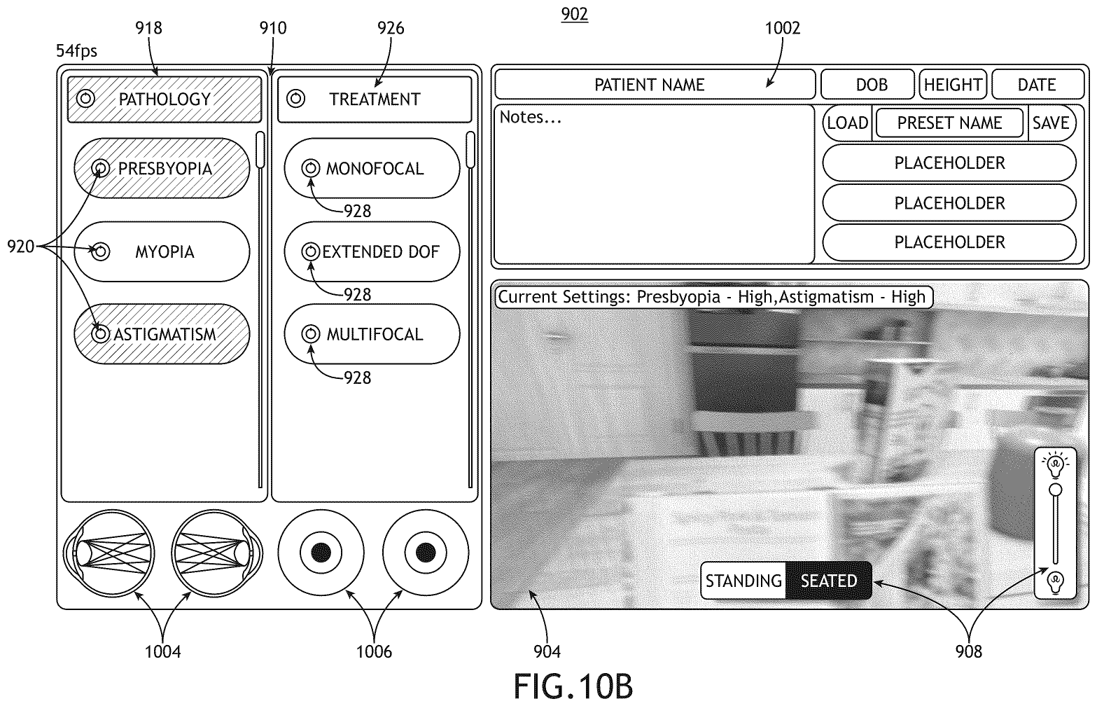

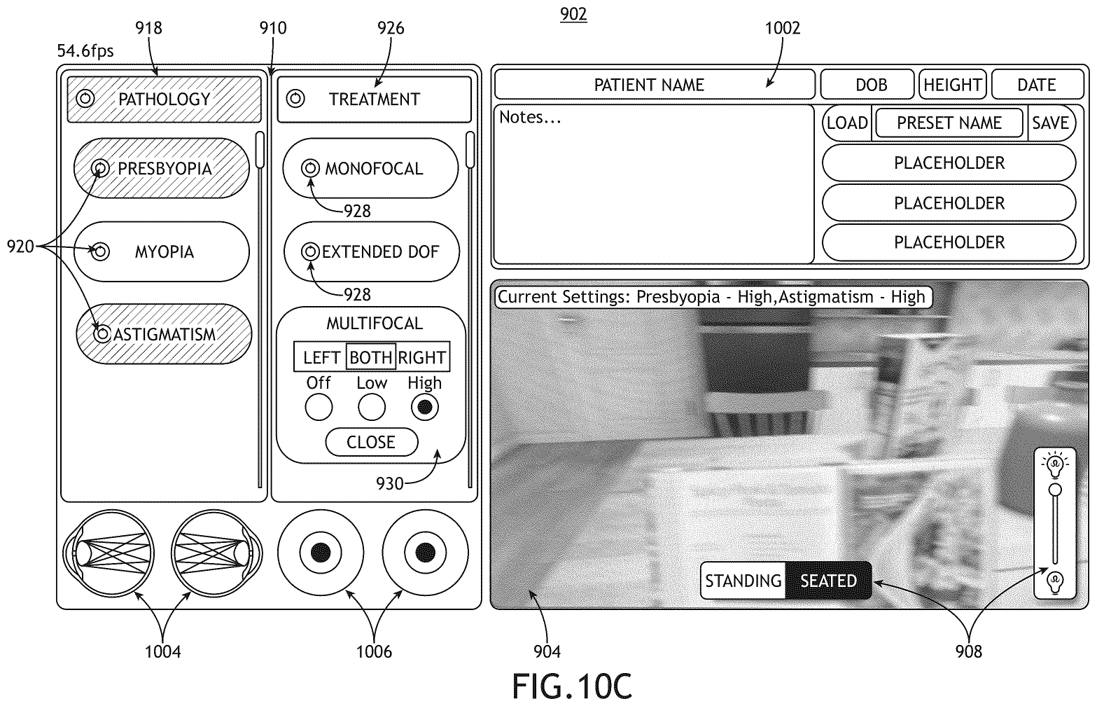

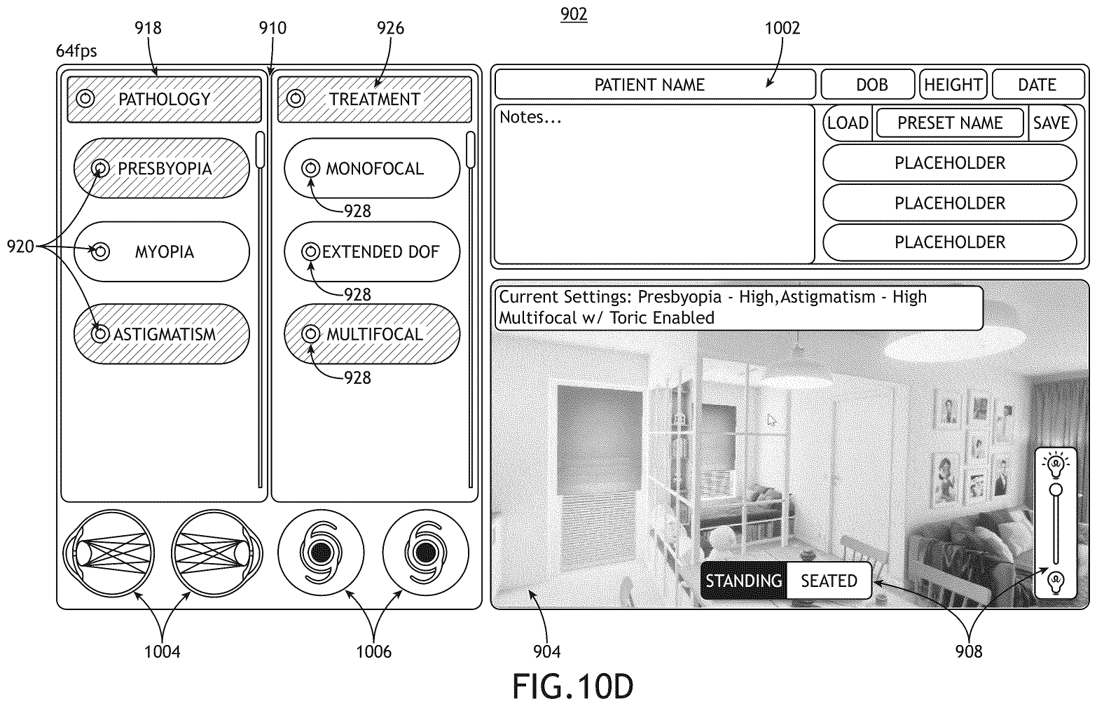

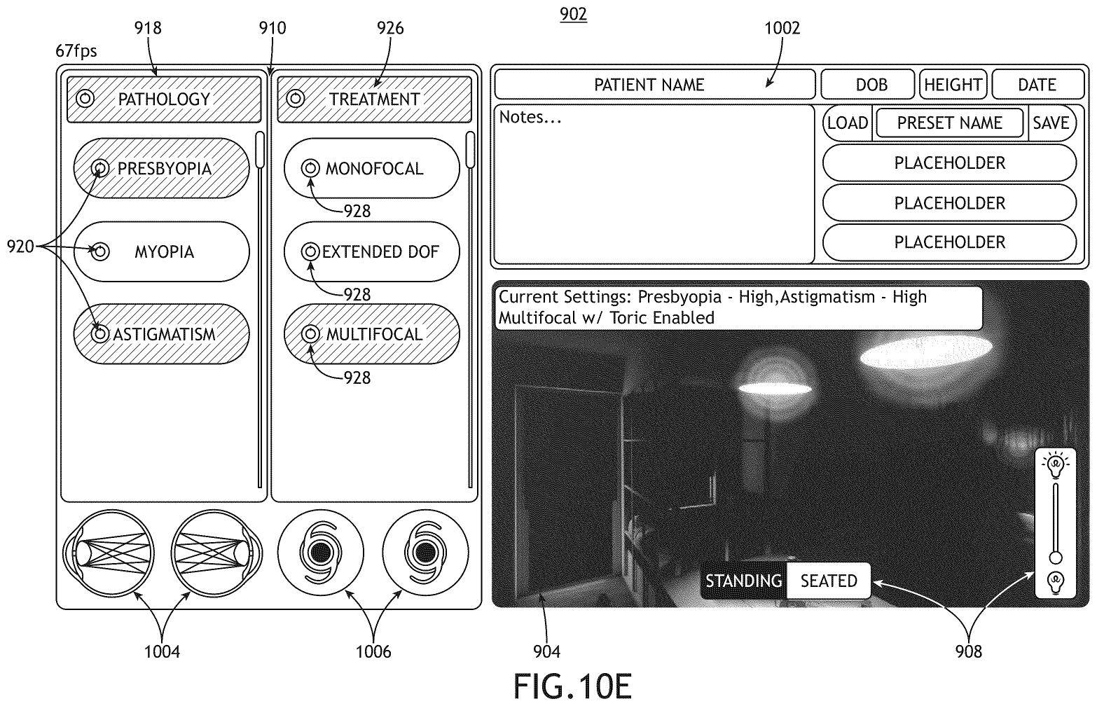

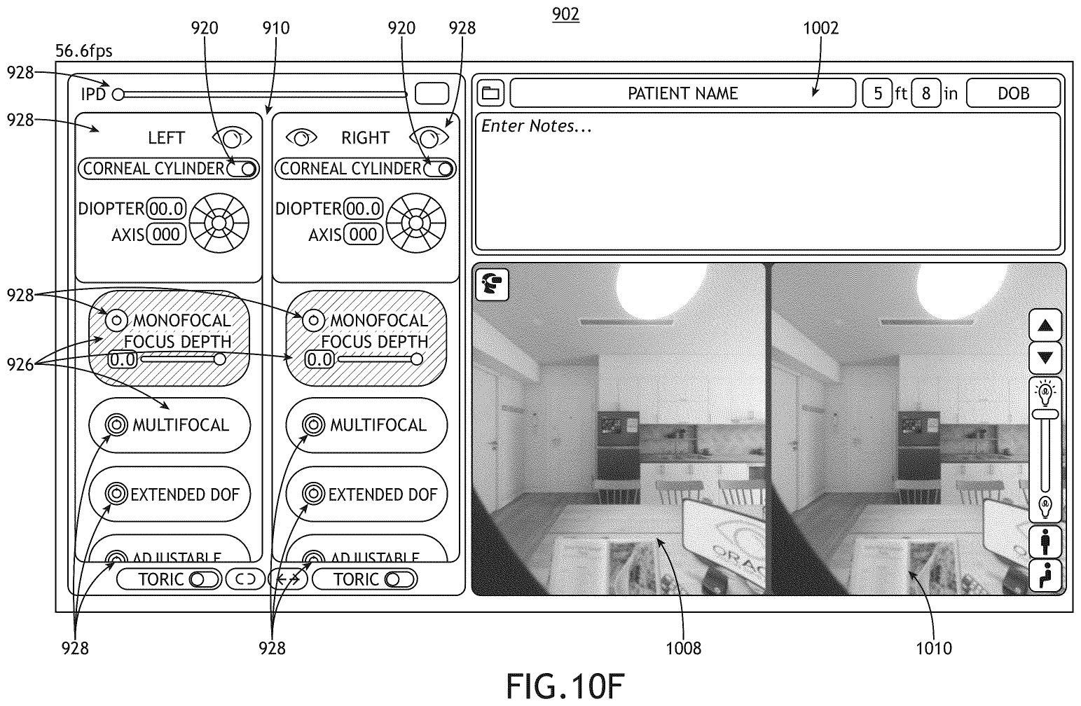

Referring now to FIGS. 9A-10E, the programmatic flow of non-limiting configuration of aspects of a user interface 112 of the VR system 100 are described in greater detail, in accordance with one or more embodiments of the present disclosure. It is contemplated herein that the VR system 100 may generally have any user interface suitable for enabling a user to selectively view any number of virtual environments with any number of visual profiles. In some embodiments, the VR system 100 provides an interactive control panel to the user to allow the user to switch between different visual profiles and/or virtual environments. For example, the user interface 112 may include the headset 102 providing a display for the user and one or more additional input devices (e.g., handheld controllers, or the like) suitable for enabling the user to make selections on content displayed via the headset 102. In some embodiments, the VR system 100 is configured to allow a third party (e.g., a clinician) to select the visual profile and/or the virtual environment to display in the headset 102. For example, the user interface 112 may display a control panel on an additional screen (e.g., a monitor, a television, a tablet device, a mobile phone, or the like) and accept user input for interacting with the control panel.

FIG. 9A illustrates a simplified block diagram depicting data flow and operation of the VR system 100, in accordance with one or more embodiments of the present disclosure. In particular, FIG. 9A depicts an embodiment in which the user interface 112 includes a clinician interface 902 that may provide information such as, but not limited to, a preview 904 (e.g., a 2D preview) of the virtual environment shown to a user via the headset 102 and one or more control panels that allow the clinician to control what the user sees in the headset 102.

The clinician interface 902 may include various control panels that allow the clinician to customize, control, and/or adjust what is displayed to the user wearing the headset 102. Further, although not explicitly shown, the clinician interface 902 may include one or more control panels for selecting whether images are display to both eyes of the user or only to one selected eye of the user. Such a control panel may include any type or design known in the art and may include, but is not limited to, buttons, list selectors, sliders, or alpha/numeric input boxes.

For example, the clinician interface 902 may include a control 906 to select the virtual environment displayed in the headset 102. As illustrations, this control 906 may allow the clinician to switch between different available virtual environments (e.g., indoor scenes, outdoor scenes, or the like) and/or select a location of the user within the virtual environment. As another example, the clinician interface 902 may include a control 908 to configure various aspects of the virtual environment such as, but not limited to, a lighting level or select whether the user is seated or standing. As another example, the clinician interface 902 may include a control 910 to select a visual profile. For instance, as will be described in greater detail with respect to FIGS. 10A-10F, the control 910 may be used to import user pathology data, adjust user pathology, select a treatment plan, or the like to provide a visual profile. Further, in some cases, one or more visual profiles may be saved in memory 108 and selected by the clinician. As another example, the clinician interface 902 may include a control 912 to toggle whether or not the visual profile is applied. For instance, the controller 104 (not shown explicitly in FIG. 9A) may render VR images of a field of view of the virtual environment from the location of the user using traditional techniques when the toggle is off (e.g., box 914), but may render VR images of a field of view of the virtual environment from the location of the user based on virtual profile selected by control 910 when the toggle is on (e.g., box 916).

FIG. 9B illustrates a simplified block diagram depicting selection of a visual profile (e.g., using control 910 in FIG. 9A), in accordance with one or more embodiments of the present disclosure.

In some embodiments, the clinician interface 902 displays various pathology controls 918 related to the user pathology (e.g., aberrations, biometric data, or the like). For example, FIG. 9B depicts three pathology controls 918 related to astigmatism, myopia, and presbyopia, though it is to be understood that this is merely illustrative and not limiting. Each of the pathology controls 918 may then include toggles 920 for whether or not they apply along with settings 922 (e.g., strength, direction, left-eye and right-eye specific settings, or the like) for each. Further, the settings 922 may be populated based on user pathology data 924 (e.g., stored in memory 108) and/or adjusted using the clinician interface 902. Any settings may then be saved again in the memory 108.

In some embodiments, the clinician interface 902 displays various treatment controls 926 related to available treatment options. For example, FIG. 9B depicts three treatment controls 926 related to a monofocal lens, a multi-focal lens, or an extended depth of field (EDoF) lens, though it is to be understood that this is merely illustrative and not limiting. Each of the treatment controls 926 may then include toggles 928 for whether or not they apply along with settings 930 (e.g., strength, direction, left-eye and right-eye specific settings, or the like) for each.

The various settings from the pathology controls 918 and the treatment controls 926 may then be used to develop a visual profile 932 for use in rendering the VR images of the virtual environment based on the flow in FIG. 9A. Further, the visual profile may be stored in the memory 108.

FIGS. 10A-10E are exemplary screenshots of a clinician interface 902, in accordance with one or more embodiments of the present disclosure. FIGS. 10A-10E include various controls depicted in FIGS. 9A and 9B, but it is to be understood that the number and type of the controls as well as their design or layout are merely illustrative and not limiting on the present disclosure. Further, FIGS. 10A-10E include additional fields 1002 for user information, pathology diagrams 1004, and treatment diagrams 1006.

FIG. 10A illustrates a first screenshot of a clinician interface 902, in accordance with one or more embodiments of the present disclosure. FIG. 10A depicts a preview 904 of a virtual environment including a kitchen in which pathology controls 918 are configured to provide presbyopia (with settings 922 not shown) and in which settings 922 for astigmatism are being configured, but not applied.

FIG. 10B illustrates a second screenshot of a clinician interface 902, in accordance with one or more embodiments of the present disclosure. FIG. 10B is substantially the same as FIG. 10A except that the pathology controls 918 are further configured with astigmatism settings applied.

FIG. 10C illustrates a third screenshot of a clinician interface 902, in accordance with one or more embodiments of the present disclosure. FIG. 10C is substantially the same as FIG. 10B except that the treatment controls 926 for a multifocal lens are opened but not applied. The preview 904 further shows a slightly different image of the virtual environment due to motion of the user.

FIG. 10D illustrates a fourth screenshot of a clinician interface 902, in accordance with one or more embodiments of the present disclosure. FIG. 10D is substantially the same as FIG. 10C except that the treatment controls 926 for a multifocal lens are applied. The preview 904 further shows the impact of this treatment option. Further, in FIG. 10D, the user is in a standing position (e.g., as selected by a virtual environment control 908).

FIG. 10E illustrates a fifth screenshot of a clinician interface 902, in accordance with one or more embodiments of the present disclosure. FIG. 10E is substantially the same as FIG. 10D except that the room lighting is adjusted (e.g., as selected by a virtual environment control 908). The preview 904 further shows the impact of the lighting change (e.g., visible ring artifacts around lights, or the like).

FIG. 10F illustrates a sixth screenshot of a clinician interface 902, in accordance with one or more embodiments of the present disclosure. FIG. 10F represents an additional non-limiting design of the clinician interface 902 configured to provide a separate left-eye VR image 1008 to a left eye of a user and a right-eye VR image 1010 to a right eye of the user. Additionally, the clinician interface 902 depicted in FIG. 10F includes separate controls 910 to manipulate the vision profile used to render the VR environment to each eye. For example, the clinician interface 902 depicted in FIG. 10F depicts separate pathology controls 918 and treatment controls 928 for each eye.

Referring again to FIG. 1, various additional aspects of the VR system 100 are described in greater detail, in accordance with one or more embodiments of the present disclosure.

The headset 102 may be communicatively coupled to the controller 104 using any suitable communication link. In some embodiments, the headset 102 is connected to the controller 104 using a wired communication link. In some embodiments, the headset 102 is connected to the controller 104 using a wireless communication link such as, but not limited to, a WiFi communication link, a Bluetooth communication link, or any radio-frequency (RF) communication link.

The one or more processors 106 of a controller 104 may include any processor or processing element known in the art. For the purposes of the present disclosure, the term “processor” or “processing element” may be broadly defined to encompass any device having one or more processing or logic elements (e.g., one or more micro-processor devices, one or more application specific integrated circuit (ASIC) devices, one or more field programmable gate arrays (FPGAs), or one or more digital signal processors (DSPs)). In this sense, the one or more processors 106 may include any device configured to execute algorithms and/or instructions (e.g., program instructions stored in memory). In one embodiment, the one or more processors 106 may be embodied as a desktop computer, mainframe computer system, workstation, image computer, parallel processor, networked computer, or any other computer system configured to execute a program configured to operate or operate in conjunction with the VR system 100, as described throughout the present disclosure.

Moreover, the processors 106 may be located in a common housing or may be distributed between multiple housings and/or between different subsystems of the VR system 100 (e.g., the headset 102, separate controllers, or the like). Therefore, the above description should not be interpreted as a limitation on the embodiments of the present disclosure but merely as an illustration. Further, the steps described throughout the present disclosure may be carried out by a single controller 104 or, alternatively, multiple controllers.

The memory 108 may include any storage medium known in the art suitable for storing program instructions executable by the associated one or more processors 134. For example, the memory 108 may include a non-transitory memory medium. By way of another example, the memory 108 may include, but is not limited to, a read-only memory (ROM), a random-access memory (RAM), a magnetic or optical memory device (e.g., disk), a magnetic tape, a solid-state drive and the like. It is further noted that the memory 108 may be housed in a common controller housing with the one or more processors 106. In one embodiment, the memory 108 may be located remotely with respect to the physical location of the one or more processors 106 and controller 104. For instance, the one or more processors 106 of the controller 104 may access a remote memory (e.g., server), accessible through a network (e.g., internet, intranet and the like).

The herein described subject matter sometimes illustrates different components contained within, or connected with, other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “connected” or “coupled” to each other to achieve the desired functionality, and any two components capable of being so associated can also be viewed as being “couplable” to each other to achieve the desired functionality. Specific examples of couplable include but are not limited to physically interactable and/or physically interacting components and/or wirelessly interactable and/or wirelessly interacting components and/or logically interactable and/or logically interacting components.

It is believed that the present disclosure and many of its attendant advantages will be understood by the foregoing description, and it will be apparent that various changes may be made in the form, construction, and arrangement of the components without departing from the disclosed subject matter or without sacrificing all of its material advantages. The form described is merely explanatory, and it is the intention of the following claims to encompass and include such changes. Furthermore, it is to be understood that the invention is defined by the appended claims.

What is claimed:

1. A system comprising:
a controller configured to be communicatively coupled to a headset, the controller including one or more processors configured to execute program instructions causing the one or more processors to:
generate a virtual environment;
receive a vision profile, wherein the vision profile includes one or more models providing vision characteristics controlling a rendering of the virtual environment;
generate one or more virtual reality (VR) images representative of the virtual environment based on the vision profile; and
provide the one or more VR images to the headset for display to a user.

2. The system of claim 1, wherein the vision characteristics comprise:
at least one of corneal aberrations, ocular biometry measurements, scotoma measurements, optical field measurements, contrast sensitivity measurements, or accommodation range.

3. The system of claim 1, wherein the vision characteristics comprise:
lens power required to produce at least one of selected emmetropic or myopic outcomes.

4. The system of claim 1, wherein the vision characteristics correspond to pre-operative vision characteristics of the user.

5. The system of claim 1, wherein the vision characteristics correspond to pre-operative characteristics of the user as corrected by a non-surgical device.

6. The system of claim 5, wherein the non-surgical device comprises:
at least one of eyeglasses or contact lenses.

7. The system of claim 1, wherein the vision characteristics correspond to post-operative vision characteristics of the user based on a selected surgical treatment.

8. The system of claim 1, wherein the vision profile is selected from a set of two or more vision profiles.

9. The system of claim 8, wherein the vision characteristics are selectable by at least one of the user or a clinician.

10. The system of claim 8, wherein the set of two or more vision profiles include:
one or more vision profiles associated with pre-operative vision characteristics of one or more eyes of the user; and
one or more vision profiles associated with post-operative vision characteristics based on one or more surgical treatments.

11. The system of claim 10, wherein the one or more surgical treatments comprise:
one or more intraocular lenses with different properties.

12. The system of claim 1, wherein the virtual environment is selectable by at least one of the user or a clinician.

13. The system of claim 1, wherein the virtual environment includes a plurality of objects at different distances from a location of the user in the virtual environment, wherein the vision characteristics provide distance-dependent visual properties, wherein representations of the plurality of objects in the one or more VR images are controlled based on associated distances from the location of the user in the virtual environment and the distance-dependent visual properties.

14. A system comprising:
a headset configured to display one or more VR images to a user; and
a controller configured to be communicatively coupled to the headset, the controller including one or more processors configured to execute program instructions causing the one or more processors to:
generate a virtual environment;
receive a vision profile, wherein the vision profile includes one or more models providing vision characteristics controlling a rendering of the virtual environment;
generate one or more virtual reality (VR) images representative of the virtual environment based on the vision profile; and
provide the one or more VR images to the headset for display to the user.

15. The system of claim 14, wherein the vision characteristics comprise:
at least one of corneal aberrations, ocular biometry measurements, scotoma measurements, optical field measurements, contrast sensitivity measurements, or accommodation range.

16. The system of claim 14, wherein the vision characteristics comprise:
lens power required to produce at least one of selected emmetropic or myopic outcomes.

17. The system of claim 14, wherein the vision characteristics correspond to pre-operative vision characteristics of the user.

18. The system of claim 14, wherein the vision characteristics correspond to pre-operative characteristics of the user as corrected by a non-surgical device.

19. The system of claim 18, wherein the non-surgical device comprises:
at least one of eyeglasses or contact lenses.

20. The system of claim 14, wherein the vision characteristics correspond to post-operative vision characteristics of the user based on a selected surgical treatment.

21. The system of claim 14, wherein the vision profile is selected from a set of two or more vision profiles.

22. The system of claim 21, wherein the vision characteristics are selectable by at least one of the user or a clinician.

23. The system of claim 21, wherein the set of two or more vision profiles include:
one or more vision profiles associated with pre-operative vision characteristics of one or more eyes of the user; and
one or more vision profiles associated with post-operative vision characteristics based on one or more surgical treatments.

24. The system of claim 23, wherein the one or more surgical treatments comprise:
one or more intraocular lenses with different properties.

25. The system of claim 14, wherein the virtual environment is selectable by at least one of the user or a clinician.

26. The system of claim 14, wherein the virtual environment includes a plurality of objects at different distances from a location of the user in the virtual environment, wherein the vision characteristics provide distance-dependent visual properties, wherein representations of the plurality of objects in the one or more VR images are controlled based on associated distances from the location of the user in the virtual environment and the distance-dependent visual properties.

27. A method comprising:
generating a virtual environment;
generating a vision profile, wherein the vision profile includes one or more models providing vision characteristics controlling a rendering of the virtual environment;
generate one or more virtual reality (VR) images representative of the virtual environment based on the vision profile; and
provide the one or more VR images to a headset for display to a user.
